(19) United States
(12) Patent Application Publication
(10) Pub. No.: US 2015/0087930 A1
(43) Pub. Date: Mar. 26, 2015

(54) METHOD AND DEVICE FOR DETERMINING SLEEPING PATTERN, AND COMPUTER READABLE STORAGE MEDIUM RECORDING THE METHOD

(71) Applicants: SAMSUNG ELECTRONICS CO., LTD., Suwon-si (KR); SEOUL NATIONAL UNIVERSITY R&DB FOUNDATION, Seoul (KR)

(72) Inventors: Jae-geol CHO, Yongin-si (KR); Byung-hun CHOI, Suwon-si (KR); Kwang-suk PARK, Seoul (KR); Hee-nam YOON, Seoul (KR); Su-hwan HWANG, Seoul (KR)

(73) Assignees: SEOUL NATIONAL UNIVERSITY R&DB FOUNDATION, Seoul (KR); SAMSUNG ELECTRONICS CO., LTD., Suwon-si (KR)

(21) Appl. No.: 14/493,806

(22) Filed: Sep. 23, 2014

(30) Foreign Application Priority Data
Sep. 23, 2013 (KR) 10-2013-0112867

(51) Int. Cl. A61B 5/00; A61B 5/11; A61B 5/08

(52) U.S. Cl. CPC A61B 5/4818; A61B 5/0803; A61B 5/11
USPC 600/301

(57) ABSTRACT

A method and apparatus for measuring a first bio-signal from a user in an unrestrictive manner are provided. The method includes extracting a second bio-signal including a motion signal and a respiration signal, based on the first bio-signal; determining an apnea measurement reference signal including a respiration measurement signal and a motion measurement signal, based on the second bio-signal; dividing the apnea measurement reference signal by a first time interval to generate a first evaluation signal, and dividing the first evaluation signal by a second time interval, that is smaller than the first time interval, to generate second evaluation signals; calculating standard deviation information based on the second evaluation signals; and determining a sleeping pattern for the second time intervals corresponding to the second evaluation signals, based on the standard deviation information.

### CROSS-REFERENCE TO RELATED APPLICATION

This application claims priority from Korean Patent Application No. 10-2013-0112867, filed on Sep. 23, 2013 in the Korean Intellectual Property Office, the disclosure of which is incorporated herein in its entirety by reference.

### BACKGROUND

1. Field

Apparatuses and methods consistent with exemplary embodiments relate to a method and device for estimating sleep apnea, and a computer readable storage medium recording the method.

2. Description of Related Art

Sleep apnea analysis in a related art measures a large number of bio-signals from a user, detects features of bio-signals generated when sleep apnea occurs, and analyzes the sleep apnea based on the features. However, various types of bio-signal measuring electrodes must be attached to the body or the face of the user in order to measure the large number of bio-signals, thereby interfering with a user's normal sleeping environment.

Also, in order to analyze the large number of the various types of measured bio-signals requires a hospital where an experienced expert is able to analyze the bio-signals. Thus, a patient must visit a hospital or other facility, and incur measurement time and costs that are relatively high.

### SUMMARY

Exemplary embodiments overcome the above disadvantages and other disadvantages not described above. Also, an exemplary embodiment is not required to overcome the disadvantages described above, and an exemplary embodiment may not overcome any of the problems described above.

One or more exemplary embodiments provide a method and device for estimating sleep apnea, which are capable of determining whether sleep apnea has occurred for a set time period by measuring bio-signals from a user in an unrestrictive manner during a normal sleep period without directly attaching any devices to the body of the user and by estimating an aspect of a change in the sizes of the bio-signals based on standard deviation information. Also provided is a computer readable storage medium having recorded thereon the method.

Additional aspects will be set forth in part in the description which follows and, in part, will be apparent from the description, or may be learned by practice of the exemplary embodiments.

According to one or more embodiments of the present invention, a method of determining a sleeping pattern includes measuring a first bio-signal from a user in an unrestrictive manner by using a poly(vinylidene difluoride) (PVDF)-based sensor having piezoelectric characteristics; extracting a second bio-signal, including a motion signal and a respiration signal, based on the first bio-signal; determining an apnea measurement reference signal, including a respiration measurement signal and a motion measurement signal, based on the second bio-signal; generating a plurality of second evaluation signals by dividing first evaluation signals, which are obtained by dividing the apnea measurement reference signal by a first time interval, by a second time interval; calculating standard deviation information including standard deviations of the plurality of second evaluation signals and an average standard deviation based on a standard deviation of a second respiration evaluation signal; and determining the sleeping pattern for the second time interval corresponding to the second evaluation signals, based on the standard deviation information, wherein the sleeping pattern includes a motion estimation section, an apnea estimation section, or a normal respiration estimation section.

The determining of the apnea measurement reference signal may include filtering the second bio-signal by using a low-pass filter; and determining the respiration measurement signal by analyzing principle components of the filtered signal, and determining the motion measurement signal by analyzing principle components of the second bio-signal.

The generating of the plurality of second evaluation signals may include generating a plurality of second motion evaluation signals by dividing a first motion evaluation signal, which is obtained by dividing the motion measurement signal by the first time interval, by the second time interval, and generating a plurality of second respiration evaluation signals by dividing first respiration evaluation signals, which are obtained by dividing the respiration measurement signal by the first time interval, by the second time interval.

The calculating of the standard deviation information may include calculating standard deviations of the plurality of second motion evaluation signals and the plurality of second respiration evaluation signals, respectively; and calculating an average of the standard deviations of the plurality of second respiration evaluation signals, except for a maximum standard deviation of the plurality of second respiration evaluation signals.

The determining of the sleeping pattern for the second time interval corresponding to the second evaluation signals may include determining the second time interval corresponding to the second evaluation signals to be the motion estimation section when the standard deviations of the second motion evaluation signals are equal to or greater than a specific value or when the standard deviations of the second respiration evaluation signals are equal to or greater than the average of the standard deviations of the second respiration evaluation signals; determining the second time interval corresponding to the second evaluation signals to be the apnea estimation section when the second time interval is not the motion estimation section and when the standard deviations of the second respiration evaluation signals are less than or equal to a product of the average of the standard deviations of the second respiration evaluation signals and ‘k’, wherein k is a real number satisfying 0<k<1; and determining the second time interval corresponding to the second evaluation signals to be the normal respiration estimation section when the second interval time is neither the motion estimation section nor the apnea estimation section.

The method may further include determining the first time interval corresponding to the first evaluation signal to be a section in which sleep apnea occurs, when at least one of sleeping patterns for a plurality of second time intervals corresponding to the plurality of second evaluation signals included in the first evaluation signals is determined to be the apnea estimation section.

When a minimum interval between the second evaluation signals determined to be the apnea estimation sections may be less than the second time interval, the method may further include determining that apnea lasts for the minimum interval.

The generating of the plurality of second evaluation signals may include generating the plurality of second evaluation signals to overlap with each other.

According to one or more embodiments of the present invention, a device for determining a sleeping pattern includes a signal measuring sensor unit having piezoelectric characteristics and configured to measure a first bio-signal from a user in an unrestrictive manner; a bio-signal extraction unit for extracting a second bio-signal, including a motion signal and a respiration signal, based on the first bio-signal measured by the signal measuring sensor unit; a reference signal determination unit for determining an apnea measurement reference signal, including a respiration measurement signal and a motion measurement signal, based on the second bio-signal extracted by the bio-signal extraction unit; an evaluation signal generation unit for generating a plurality of second evaluation signals by dividing first evaluation signals by a second time interval, wherein the first evaluation signals are obtained by dividing the apnea measurement reference signal, which is determined by the reference signal determination unit, by a first time interval; a standard deviation information calculation unit for calculating standard deviation information which includes standard deviations of the plurality of second evaluation signals determined by the evaluation signal generation unit and an average standard deviation based on a standard deviation of a second respiration evaluation signal; and a sleeping pattern determination unit for determining a sleeping pattern for the second time interval corresponding to the second evaluation signals, based on the standard deviation information calculated by the standard deviation information calculation unit, wherein the sleeping pattern includes a motion estimation section, an apnea estimation section, or a normal respiration estimation section.

The reference signal determination unit may include a low-pass filter unit for filtering the second bio-signal; and a principle component analysis unit for determining the respiration measurement signal by analyzing principle components of the filtered signal, and determining the motion measurement signal by analyzing principle components of the second bio-signal.

The evaluation signal generation unit may include a first time division unit for generating first motion evaluation signals by dividing the motion measurement signal by a first time interval, and generating first respiration evaluation signals by dividing the respiration measurement signal by the first time interval; and a second time division unit for generating a plurality of second motion evaluation signals by dividing the first motion evaluation signals by a second time interval, and generating a plurality of second respiration evaluation signals by dividing the first respiration evaluation signals by the second time interval.

The standard deviation information calculation unit may calculate standard deviations of the plurality of second motion evaluation signals and the plurality of second respiration evaluation signals, respectively, and calculate an average of the standard deviations of the plurality of second respiration evaluation signals, except for a maximum standard deviation of the plurality of second respiration evaluation signals

The sleeping pattern determination unit may determine the sleeping pattern for the second time interval corresponding to the second evaluation signals to be the motion estimation section when the standard deviations of the second motion evaluation signals are equal to or greater than a specific value or when the standard deviations of the second respiration evaluation signals are equal to or greater than the average of the standard deviations of the second respiration evaluation signals; determine the sleeping pattern for the second time interval corresponding to the second evaluation signals to be the apnea estimation section when the sleeping pattern is not the motion estimation section and when the standard deviations of the second respiration evaluation signals are less than or equal to a product of the average of the standard deviations of the second respiration evaluation signals and ‘k’, wherein k is a real number satisfying 0<k<1; and determine the sleeping pattern for the second time interval corresponding to the second evaluation signals to be the normal respiration estimation section when the sleeping pattern is neither the motion estimation section nor the apnea estimation section.

The device may further include an apnea generation determination unit for determining the first time interval corresponding to the first evaluation signals to be a section in which apnea occurs when at least one apnea estimation section determined by the sleeping pattern determination unit is present in a plurality of second time intervals corresponding to the plurality of second evaluation signals included in the first evaluation signals.

When a minimum interval between apnea estimation sections determined for the first time interval is equal to the second time interval, the sleeping pattern determination unit may determine the minimum interval to be the apnea estimation section.

The evaluation signal generation unit may generate the plurality of second evaluation signals to overlap with each other.

### BRIEF DESCRIPTION OF THE DRAWINGS

The and/or other aspects will become apparent and more readily appreciated from the following description of exemplary embodiments, taken in conjunction with the accompanying drawings in which:

FIG. 1A is a diagram illustrating bio-signal measuring electrodes of a related art, and FIG. 1B is a diagram illustrating a signal measuring sensor for measuring sleep apnea, according to an exemplary embodiment;

FIG. 2 is a flowchart of a method of estimating sleep apnea, according to an exemplary embodiment;

FIG. 3 is a block diagram of a device for estimating sleep apnea, according to an exemplary embodiment;

FIG. 4 is a block diagram of a bio-signal extractor according to an exemplary embodiment;

FIG. 5 is a diagram illustrating a device for estimating sleep apnea, according to an exemplary embodiment;

FIG. 6 is a diagram illustrating a method of generating a plurality of second evaluation signals, which is included in a method of estimating sleep apnea, according to an exemplary embodiment;

FIG. 7A and FIG. 7B is a flowchart of a method of determining a sleeping pattern according to an exemplary embodiment;

FIG. 8 is a diagram illustrating a method of determining a section in which sleep apnea occurs, according to an exemplary embodiment;

FIG. 9 is a diagram illustrating a method of generating second evaluation signals to overlap with each other, according to an exemplary embodiment; and

FIG. 10 is a diagram illustrating a method of determining that apnea lasts, according to an exemplary embodiment.

### DETAILED DESCRIPTION

Reference will now be made to exemplary embodiments which are illustrated in the accompanying drawings, wherein like reference numerals refer to like elements throughout. In this regard, the exemplary embodiments may have different forms and should not be construed as being limited to the descriptions set forth herein. Accordingly, the exemplary embodiments are merely described below, by referring to the figures, to explain aspects of the present description.

In the present disclosure, general terms that have been widely used are selected, if possible, in consideration of functions of the present description, but non-general terms may be selected according to the intentions of technicians in the art, precedents, or new technologies, and the like. Also, some terms may be arbitrarily chosen. In this case, the meanings of these terms is further explained in corresponding parts of the present disclosure. Thus, the terms used herein should be defined not based on the names thereof but based on the meanings thereof and the whole context of the present disclosure.

In the present disclosure, it should be understood that the terms, such as include or have, and the like, are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

As used herein, the term “and/or” includes any and all combinations of one or more of the associated listed items. Expressions such as “at least one of,” when preceding a list of elements, modify the entire list of elements and do not modify the individual elements of the list.

Hereinafter, the exemplary embodiments are described with reference to the accompanying drawings so that those of ordinary skill in the art can easily accomplish them. The present description can be, however, embodied in many various forms and are not limited to the embodiments set forth herein. Also, parts that are not related to describing the present description are omitted for clarity.

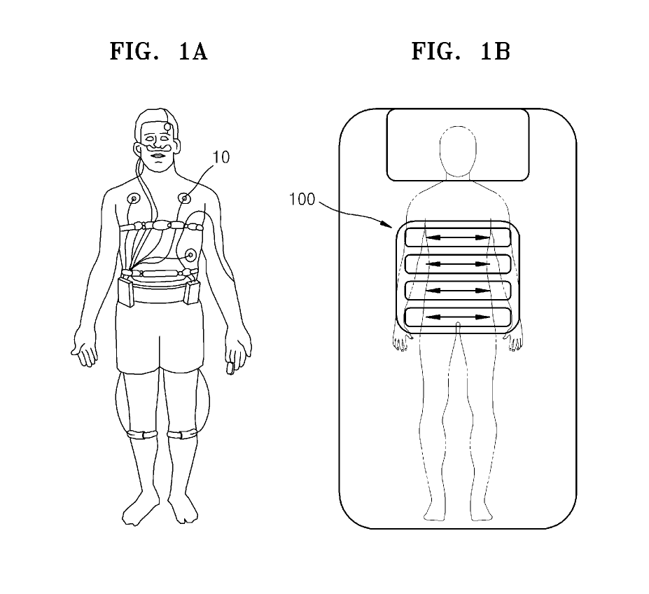

FIG. 1A is a diagram illustrating bio-signal measuring electrodes of a related art, and FIG. 1B is a diagram illustrating a signal measuring sensor 100 for measuring sleep apnea, according to an exemplary embodiment.

As illustrated in FIG. 1A, for sleep apnea analysis according to a related art, various types of the bio-signal measuring electrodes 10 are attached to the body and/or the face of a user in order to measure a large number of bio-signals. In this example, an area of the user's activity is limited, thus interfering with the user's normal sleeping environment when the bio-signal measuring electrodes 10 are attached to the user.

In contrast, referring to FIG. 1B, the signal measuring sensor 100 according to an exemplary embodiment may be installed on a bed or other surface to measure a first bio-signal from a user that is sleeping.

For example, the signal measuring sensor 100 according to an exemplary embodiment may use a poly(vinylidene difluoride) (PVDF)-based sensor having piezoelectric characteristics. Polyvinylidene difluoride is also referred to as polyvinylidene fluoride. As an example, the signal measuring sensor unit 100 may be installed in a space between the back of the user and a bed mattress.

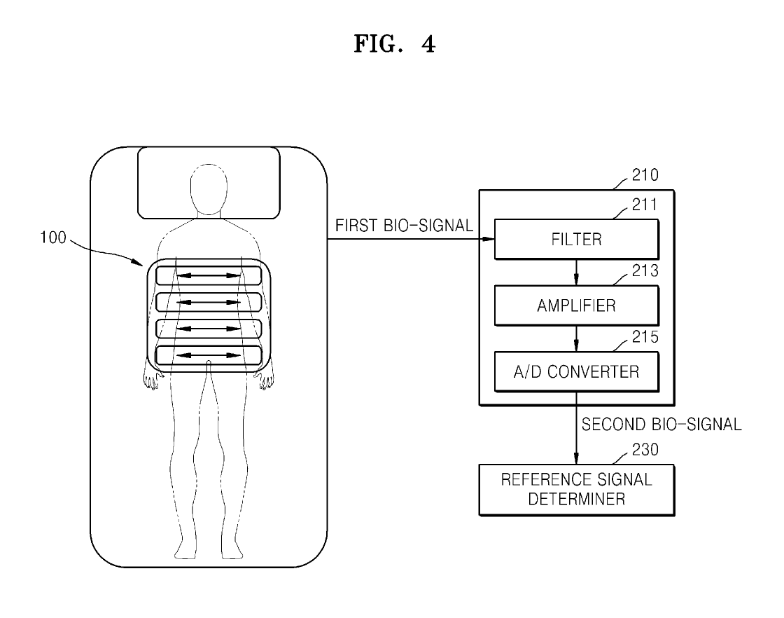

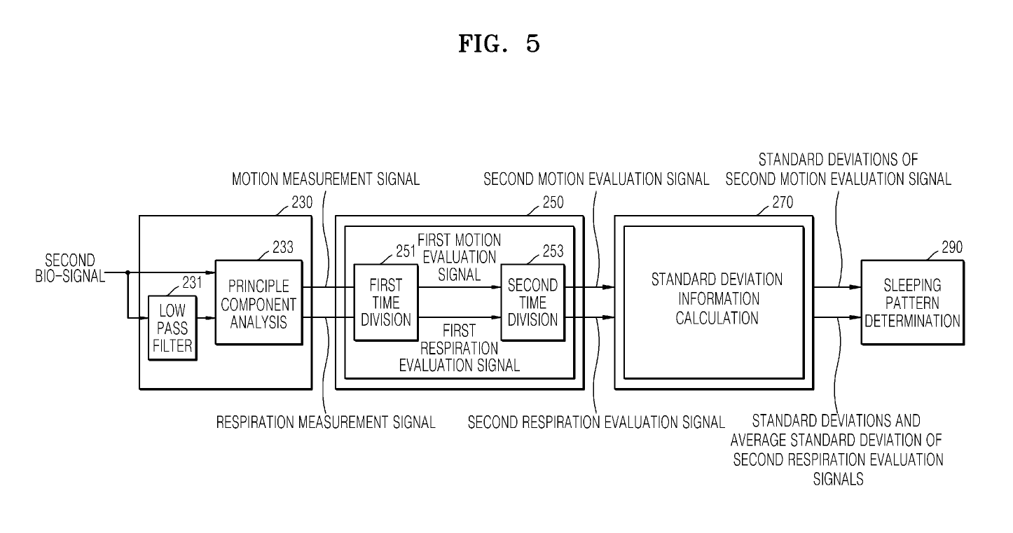

For example, the signal measuring sensor 100 may have a thickness of about 0.1 mm and is thus capable of measuring a respiration signal from the user in an unconscious state or in a sleeping state in which the user does not recognize the installation of the signal measuring sensor 100. Because a measurement position may change due to the user's movement while the user is sleeping, the signal measuring sensor 100 may have a size of, for example, 30×30 cm, and the like, to cover the user's upper body. A silicon pad having a thickness of about 1 mm may be installed on the signal measuring sensor 100 to prevent the signal measuring sensor 100 from being damaged by physical pressure. A method of generating signals to be used to measure sleep apnea from the first bio-signal is further described with reference to FIGS. 4 and 5.

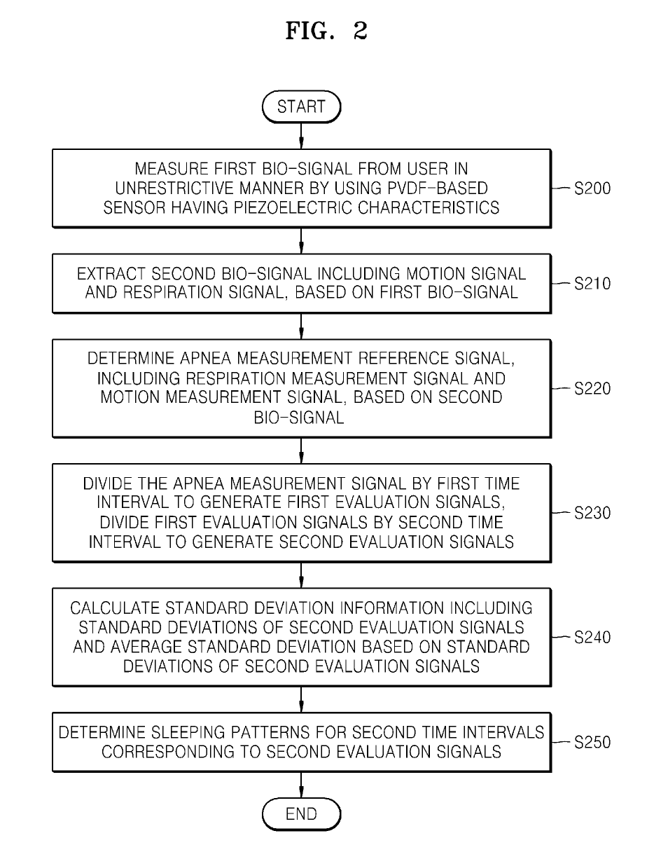

FIG. 2 is a flowchart of a method of estimating sleep apnea, according to an exemplary embodiment.

In the method of FIG. 2, in operation S200, a first bio-signal is measured from a user in an unrestrictive manner by using the signal measuring sensor 100 having piezoelectric characteristics of FIG. 1. For example, the signal measuring sensor 100 may use a PVDF-based sensor.

The signal measuring sensor 100 may be installed in a space between the user's back and a bed mattress to measure the first bio-signal without causing the user to directly contact the signal measuring sensor 100. For example, the first bio-signal may be any of various signals that may be measured using the signal measuring sensor 100.

In operation S210, a second bio-signal including a motion signal and a respiration signal is extracted based on the measured first bio-signal. For example, in a method of estimating sleep apnea, changes in the respiration signal and the motion signal may be estimated based on standard deviation information. Thus, the respiration signal and the motion signal may be extracted from the first bio-signal to perform the estimating.

Thus, the second bio-signal which is to be used to estimate sleep apnea may be generated by extracting the respiration signal and the motion signal that have specific frequency bands from the first bio-signal, amplifying the extracted signals, and performing analog/digital (A/D) conversion on the amplified signals, an example of which is described with reference to FIG. 4.

In operation S220, an apnea measurement reference signal that includes a respiration measurement signal and a motion measurement signal may be determined based on the extracted second bio-signal. For example, in operation S220 the second bio-signal may be filtered using a low-pass filter. The respiration measurement signal may be determined by analyzing principle components of a signal obtained by filtering the second bio-signal and the motion measurement signal may be determined by analyzing principle components of the second bio-signal, an example of which is described with reference to FIG. 5.

In operation S230, the apnea measurement reference signal is divided by a first time interval to generate one or more first evaluation signals, and second evaluation signals are generated by dividing the first evaluation signals by a second time interval.

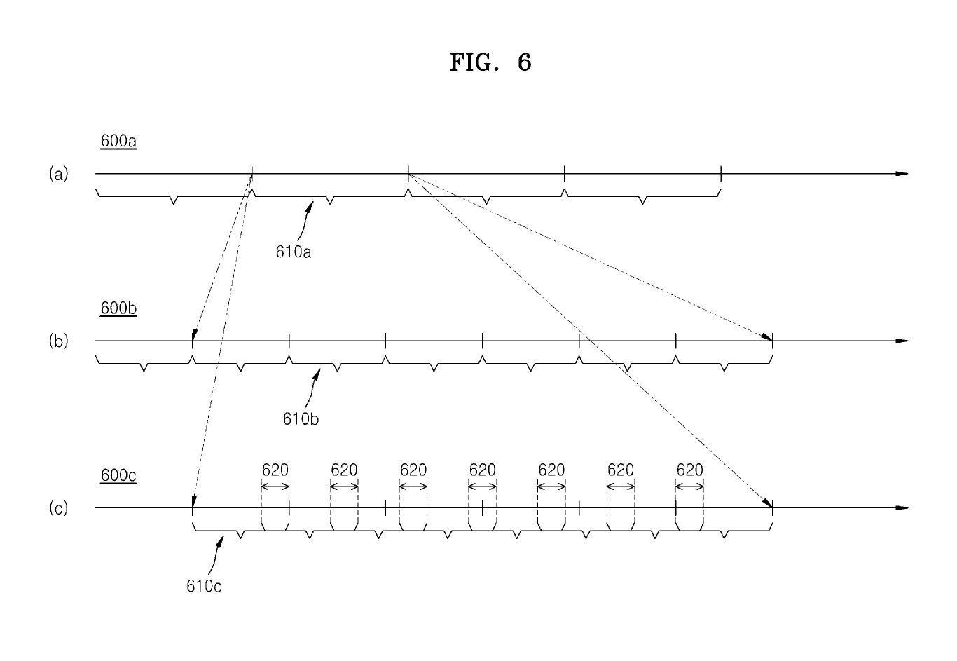

For example, the apnea measurement signal may be divided by a first time interval to generate one or more first motion evaluation signals and first respiration evaluation signals. Then, second motion evaluation signals may be generated by dividing the first motion evaluation signals, by the second time interval, and second respiration evaluation signals may be generated by dividing the first respiration evaluation signals by the second time interval. The method of FIG. 2 may further include generating second evaluation signals to overlap with each other, as will be described with reference to FIG. 6.

In operation S240, standard deviation information, including standard deviations of the second evaluation signals and an average standard deviation based on the standard deviations, is calculated. For example, the method may include calculating standard deviations of the second motion evaluation signals and second respiration evaluation signals, and calculating an average of the standard deviations of the second respiration evaluation signals except for a maximum standard deviation among the calculated standard deviations.

In operation S250, sleeping patterns for the second time intervals corresponding to the second evaluation signals are determined based on the calculated standard deviation information. For example, a sleeping pattern may include a motion estimation section, an apnea estimation section, a normal respiration estimation section, and the like.

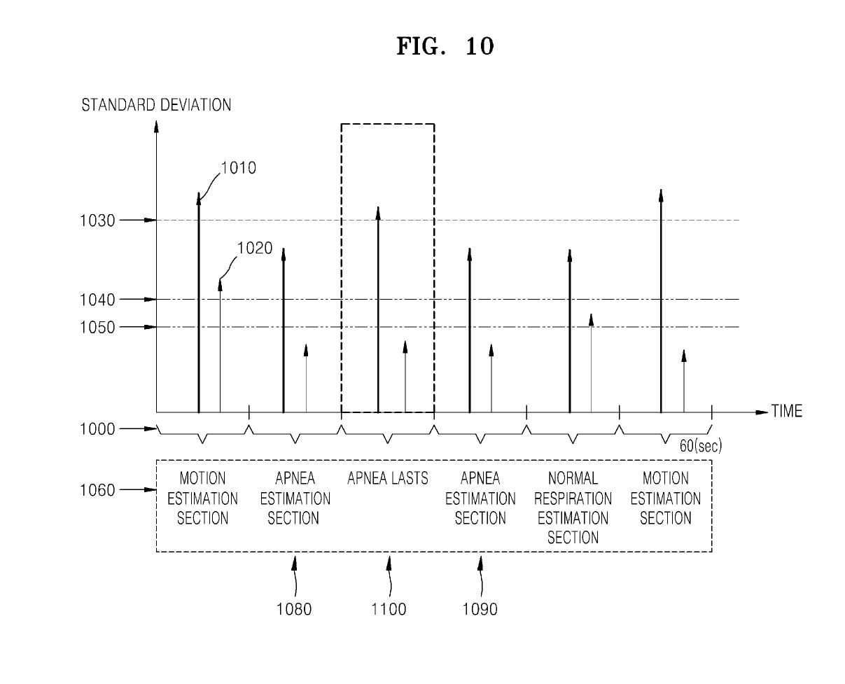

When at least one sleeping pattern among sleeping patterns for second time intervals corresponding to the second evaluation signals is determined to be an apnea estimation section, the first time interval corresponding to the first evaluation signal may be determined to be a sleep apnea generation section. When a minimum interval between the second evaluation signals determined as the apnea estimation section is less than or equal to the second time interval, it may be determined that apnea lasts for the second time interval, as is described with reference to FIG. 10.

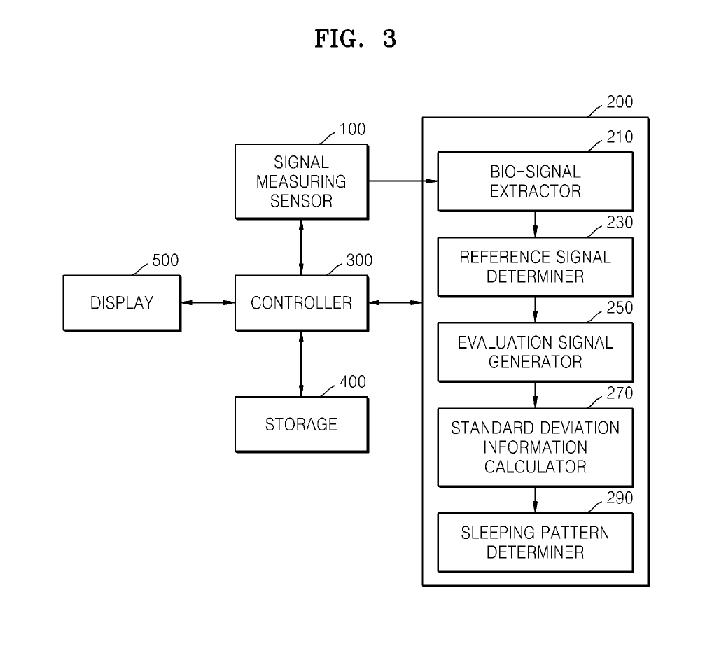

FIG. 3 is a block diagram of a device for estimating sleep apnea, according to an exemplary embodiment.

As illustrated in FIG. 3, the device for estimating sleep apnea includes a storage 400, a signal measuring sensor 100, a bio-signal extractor 210, a reference signal determiner 230, an evaluation signal generator 250, a standard deviation information calculator 270, a sleeping pattern determiner 290, a controller 300, and a display 500. However, these elements are not indispensable elements. Also, the device for estimating sleep apnea may further include other elements or may include only some of the elements illustrated in FIG. 3.

The signal measuring sensor 100 measures a first bio-signal from a user in an unrestrictive manner. According to various aspects, the first bio-signal is measured using a PVDF-based sensor that measures a signal without being attached to the body of a user. Accordingly, the user's sleep is not interfered with by the measuring of the first bio-signal.

The bio-signal extractor 210 extracts a second bio-signal, including a motion signal and a respiration signal, based on the first bio-signal measured by the signal measuring sensor 100. Because the first bio-signal may include signals measured while the user is sleeping, the second bio-signal may be used in a method of estimating sleep apnea. The second bio-signal may include the respiration signal and the motion signal, as is described with reference to FIG. 4.

The reference signal determiner 230 determines an apnea measurement reference signal, including a respiration measurement signal and a motion measurement signal, based on the second bio-signal extracted by the bio-signal extractor 210. For example, the respiration measurement signal and the motion measurement signal may be generated based on the second bio-signal to secure the quality of the motion signal and the respiration signal included in the second bio-signal and to increase the reliability of measuring sleep apnea.

Thus, a user's sleeping pattern (e.g., motion, apnea, or normal respiration, and the like) may be determined based on the respiration measurement signal and the motion measurement signal. The structure of the reference signal determiner 230 is described with reference to FIG. 5.

The evaluation signal generator generates first evaluation signals by dividing the apnea measurement signal by a first time interval. The evaluation signal generator 250 generates second evaluation signals by dividing first evaluation signals by a second time interval, as is described with reference to FIGS. 5 and 6.

The standard deviation information calculator 270 calculates standard deviation information including standard deviations of the second evaluation signals generated by the evaluation signal generation unit 250 and an average of the standard deviations. The structure of the standard deviation information calculator 270 is described with reference to FIG. 5 below.

The sleeping pattern determiner 290 determines a sleeping pattern for the second time interval corresponding to the second evaluation signals, based on the standard deviation information calculated by the standard deviation information calculator 270. For example, the sleeping pattern may include a motion estimation section, an apnea estimation section, or a normal respiration estimation section.

For example, he sleeping pattern determiner 290 may determine a sleeping pattern for the second time interval corresponding to the second evaluation signals to be the motion estimation section when a standard deviation of a second motion evaluation signal is equal to or greater than a specific value (e.g., 2) or the standard deviations of the second respiration evaluation signals are equal to or greater than the average of the standard deviations of the second respiration evaluation signals among the standard deviations. If the sleeping pattern is determined not to be the motion estimation section, and the standard deviation of the second respiration evaluation signal is less than or equal to a product of the average of the standard deviations of the second respiration evaluation signals and ‘k’ (0<k<1, for example, k=0.7), the sleeping pattern for the second time interval corresponding to the second evaluation signals may be determined to be the apnea estimation section. If the sleeping pattern is neither the motion estimation section nor the apnea estimation section, the sleeping pattern for the second time interval corresponding to the second evaluation signals may be determined to be the normal respiration estimation section.

When at least one apnea estimation section is determined by the sleeping pattern determination unit 290 for the second time intervals corresponding to the second evaluation signals included in the first evaluation signal, a sleeping pattern for the first time interval corresponding to the first evaluation signal may be determined as a section in which an apnea occurs.

When a minimum interval between apnea estimation sections determined for the first time interval is the second time interval, the sleeping pattern determiner 290 may determine the minimum interval to be the apnea estimation section.

The storage 400 may store a program for performing processing or controlling by the controller 300, and input/output data, for example, the first bio-signal, the second bio-signal, the apnea measurement reference signal, the first evaluation signal, the second evaluation signals, the standard deviation information, the sleeping pattern, and the like.

The storage 400 may include at least one storage medium selected from the group consisting of a flash memory type, a hard disk type, a multimedia card micro type, a card type memory (e.g., secure digital (SD) or extreme digital (XD) memory, etc.), random access memory (RAM), static RAM (SRAM), read-only memory (ROM), an electrically erasable programmable read-only memory (EEPROM), a programmable ROM (PROM) magnetic memory, a magnetic disc, an optical disc, and the like. Also, the device for estimating sleep apnea may operate a web storage for performing a storing function of the storage 400 via the Internet such as a cloud service.

The display 500 displays and outputs information processed by the device for estimating sleep apnea. For example, the display 500 may display various types of data input to/output from the device for estimating sleep apnea such as the first bio-signal, the second bio-signal, the apnea measurement reference signal, the first evaluation signal, the second evaluation signals, the standard deviation information, the sleeping pattern, and the like.

When a layered structure including the display 500 and a touch pad (not shown) are combined to form a touch screen, the display 500 may be used as both an output device and an input device. For example, the display 500 may include at least one selected from the group consisting of a liquid crystal display (LCD), a thin-film transistor-LCD, an organic light-emitting diode display, a flexible display, a three-dimensional (3D) display, an electrophoretic display, and the like. Also, the device for estimating sleep apnea may include two or more displays 500 according to the type thereof. In this case, the two or more displays 500 may be disposed opposite to each other via a hinge.

In general, the controller 300 controls overall operations of the device for estimating sleep apnea. For example, the controller 300 may execute programs stored in the storage 400 to control overall operations of the signal measuring sensor 100, the bio-signal extractor 210, the reference signal determiner 230, the evaluation signal generator 250, the standard deviation information calculator 270, the sleeping pattern determiner 290, the display 500, and the like.

FIG. 4 is a block diagram of the bio-signal extractor 210 according to an exemplary embodiment.

As illustrated in FIG. 4, the bio-signal extractor 210 includes a filter 211, an amplifier 213, and an A/D converter 215.

The filter 211 extracts a frequency-band signal including a respiration signal and a motion signal, from a first bio-signal measured by the signal measuring sensor 100.

A second bio-signal including a respiration signal and a motion signal, which is used in a method of estimating sleep apnea, is extracted from the first bio-signal including all bio-signals measured by the signal measurement sensor unit 100. For example, a low-pass filter may be used to extract the frequency-band signal including the respiration signal and the motion signal, from the first bio-signal. As an example, the low-pass filter may be a Butterworth low-pass filter but is not limited thereto.

The amplifier 231 amplifies the frequency-band signal, including the respiration signal and the motion signal that is output from the filter 211. For example, a gain of the amplifier 231 may be 50 but is not limited thereto.

The A/D converter 215 is used during a pre-processing process of performing digital signal processing on the first bio-signal at a later time.

The second bio-signal, including the motion signal and the respiration signal, which is output from the bio-signal extractor 210, is input to the reference signal determiner 230, as described with reference to FIG. 5.

FIG. 5 is a diagram illustrating signals input to/output from a device for estimating sleep apnea according to an exemplary embodiment.

As illustrated in FIG. 5, signal determiner 230 may include a low-pass filter 231 and a principle component analyzer (PCA) 233. For example, the reference signal determiner 230 may include the low-pass filter 231 and the PCA 233 to secure the quality of a motion signal and a respiration signal included in a second bio-signal and to increase the reliability of measuring sleep apnea. Thus, an apnea measurement reference signal may be generated and may include a respiration measurement signal and a motion measurement signal which may be used in a method of estimating sleep apnea.

The low-pass filter 231 is used to secure the quality of the respiration signal included in the second bio-signal. For example, if a cut-off frequency band of the low-pass filter 231 is 0.5 Hz or less, it may be understood that a user breaths once every two seconds when the cut-off frequency band is converted into time. Thus, when the second bio-signal is filtered using the low-pass filter 231, the respiration signal indicating that it takes two seconds or more for the user to breathe once is extracted.

The respiration measurement signal may be determined using the PCA 233, based on the respiration signal output from the low-pass filter 231. The motion measurement signal may be determined using the PCA 233, based on the second bio-signal. The respiration measurement signal and the motion measurement signal may be input to the evaluation signal generator 250.

As illustrated in FIG. 5, the evaluation signal generator 250 may include a first time division 251 and a second time division 253. For example, the evaluation signal generator 250 may use the first time division 251 and the second time division 253 to determine whether apnea occurs during sleep in units of a first time interval, based on the respiration measurement signal and the motion measurement signal.

For example, the first time interval may be one minute. In order to determine whether apnea occurs during sleep in units of the first time interval, the first time division unit 251 may generate a first respiration evaluation signal and a first motion evaluation signal by dividing the respiration measurement signal and the motion measurement signal by the first time interval of one minute.

In this case, a sleeping pattern such as a motion estimation section, an apnea estimation section, a normal respiration estimation section, and/or the like, may be determined by dividing the first respiration evaluation signal and the first motion evaluation signal by a second time interval, for example, ten seconds. The first respiration evaluation signal and the first motion evaluation signal may be divided by ten seconds based on the definition of sleep apnea in which pauses in breathing can last for ten seconds or more during sleep. Thus, the second time division unit 253 may generate a plurality of second respiration evaluation signals and a plurality of second motion evaluation signals by dividing the first respiration evaluation signal and the first motion evaluation signal by the second time interval. As another example, the plurality of second evaluation signals may be generated to overlap with each other, as is described with reference to FIG. 6.

A sleeping pattern of the user may be determined in units of the second time interval, based on standard deviation information of the plurality of second respiration evaluation signals and the plurality of second motion evaluation signals. Whether apnea occurs for the first time interval may be determined based on a result of determining a sleeping pattern in units of the second time interval.

As illustrated in FIG. 5, the standard deviation information calculator 270 may calculate standard deviation information to be used to determine a sleeping pattern in units of the second time interval. For example, the standard deviation information may include standard deviations of the plurality of respective second motion evaluation signals and the plurality of respective second respiration evaluation signals and an average of the standard deviations of the second respiration evaluation signals.

Thus, a sleeping pattern including one or more of the motion estimation section, the apnea estimation section, the normal respiration estimation section, and the like, for the first time interval may be determined in units of the second time interval, based on the standard deviation information. For example, a maximum standard deviation among standard deviations in six sections may be obtained by dividing a one minute first interval by ten second intervals.

As another example, when the average of the standard deviations of the second respiration evaluation signals is calculated, an average of the standard deviations, excluding a maximum value or a plurality of maximum values from among the standard deviations calculated for a plurality of second time intervals included in the first time interval may be calculated as a reference for determining a sleeping pattern. This may prevent the average of the standard deviations from being influenced by a standard deviation in a section in which motion occurs.

The sleeping pattern determiner 290 may receive the standard deviation information from the standard deviation information calculator 270, and determine a sleeping pattern in units of the second time interval. In this case, whether apnea occurs during the first time interval may be determined based on the sleeping patterns determined in refined units of the second time interval.

FIG. 6 is a diagram illustrating a method of generating a plurality of second evaluation signals, which is included in a method of estimating sleep apnea, according to an exemplary embodiment.

As illustrated in FIG. 6(a), a plurality of first evaluation signals 610a that include first motion evaluation signals, first respiration evaluation signals, and the like, may be generated by dividing an apnea measurement reference signal 600a that include a motion measurement signal, a respiration measurement signal, and the like by a first time interval, e.g., one minute. As illustrated in FIG. 6(b), a plurality of second evaluation signals 610b that include second motion evaluation signals, second respiration evaluation signals, and the like, are generated by dividing a first evaluation signal 600b by a second time interval, e.g., ten seconds.

Also, as illustrated in FIG. 6(c), a plurality of second evaluation signals 610c that include second motion evaluation signals, second respiration evaluation signals, and the like, are generated by dividing a first evaluation signal 600c by a second time interval, e.g., ten seconds, to overlap with each other. The number of second evaluation signals 610c generated from the first evaluation signal 600c may vary according to overlapping time sections 620.

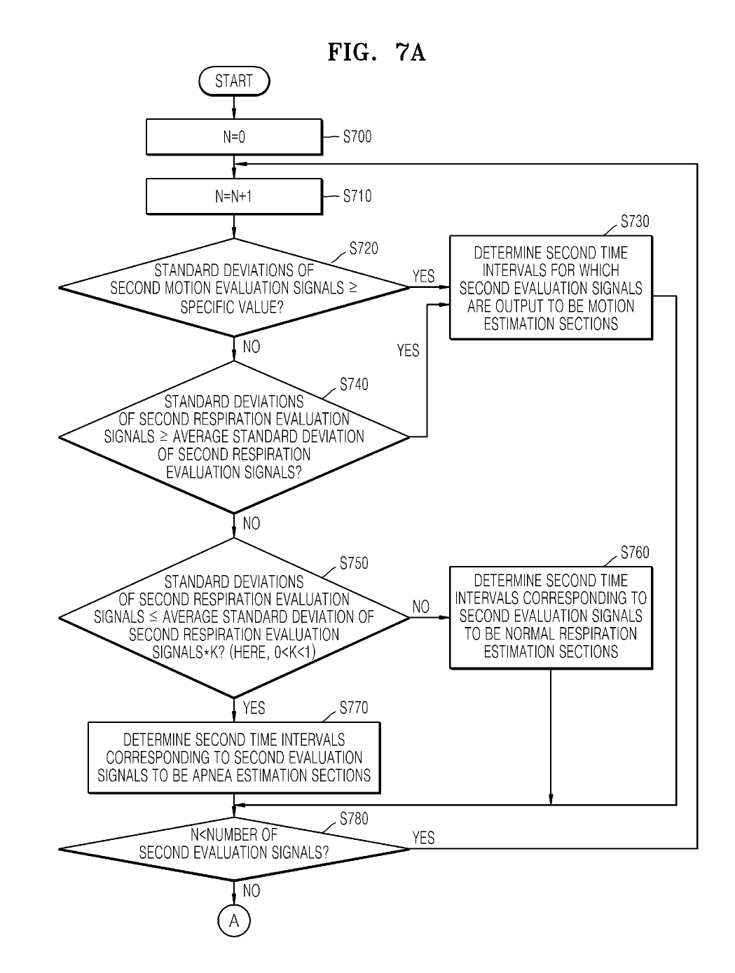

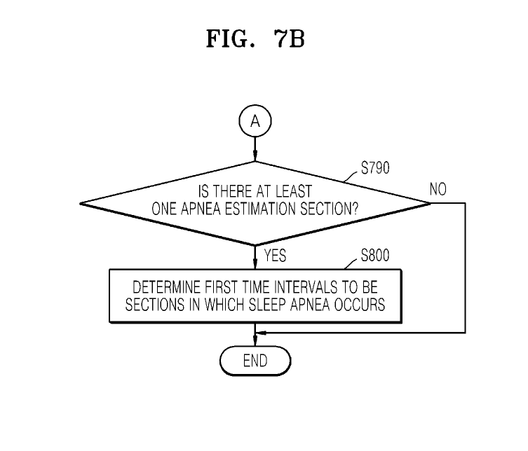

FIG. 7A and FIG. 7B is a flowchart of a method of determining a sleeping pattern, according to an exemplary embodiment.

In the method of determining a sleeping pattern, whether sleep apnea occurs during a first time interval may be estimated by analyzing first evaluation signals obtained by dividing an apnea measurement reference signal by the first time interval.

For example, a plurality of second evaluation signals obtained by subdividing the first evaluation signals by a second time interval, e.g., ten seconds, may be analyzed. Here, the reason why the first evaluation signals are subdivided by ten seconds is based on the definition of sleep apnea, i.e., pauses in breathing that can last for ten seconds or more during sleeping.

In this case, the first evaluation signals for the first time interval may be determined to be a plurality of sleeping patterns, based on standard deviation information of the plurality of respective second evaluation signals. When at least one of the plurality of sleeping patterns is determined to be an apnea estimation section in a second evaluation signals, the first time intervals corresponding to the second evaluation signals may likewise be determined to be sections in which sleep apnea occurs. Accordingly, when at least one apnea estimation section is present, the first time interval is determined to be a section in which sleep apnea occurs but the present disclosure is not limited to the number of apnea estimation sections.

Thus, the number of sleeping patterns for the first time intervals may be determined to be equal to the number of second evaluation signals.

For example, a sleeping pattern may be determined for each second evaluation signal generated from the first evaluation signals. Here, ‘N’ denotes the number of sleeping patterns determined for the first time intervals. To start with, in operation S700, N is zero. That is, operation S700 is performed to set N=0 so as to analyze a first evaluation signal for a new time. It should be appreciated that the number of sleeping patterns may vary according to the lengths of the first time interval and the second time interval. Also, when the plurality of second evaluation signals overlap with each other, the number of second evaluation signals generated from the first evaluation signals may vary according to time sections in which the plurality of second evaluation signals overlap with each other.

In operation S710, N=N+1, in which a sleeping pattern is evaluated for each second evaluation signal. For example, the plurality of second evaluation signals may each include a second respiration evaluation signal and a second motion evaluation signal.

In operation S720, it is determined whether standard deviations of the second motion evaluation signals are equal to or greater than a specific value.

If it is determined in operation S720 that the standard deviations of the second motion evaluation signals are equal to or greater than the specific value, in operation S730 the second time intervals for which the second evaluation signals are output are determined to be motion estimation sections. Alternatively, if it is determined in operation S720 that the standard deviations of the second motion evaluation signals are not equal to or greater than the specific value, in operation S740 it is determined whether standard deviations of the second respiration evaluation signals are equal to or greater than an average standard deviation of the second respiration evaluation signals.

If it is determined in operation S740 that the standard deviations of the second respiration evaluation signals are equal to or greater than the average standard deviation of the second respiration evaluation signals, the method proceeds to operation S730 and the second time intervals for which the second evaluation signals are output are determined to be motion estimation sections.

Alternatively, if it is determined in operation S740 that the standard deviations of the second respiration evaluation signals are not equal to or greater than the average standard deviation of the second respiration evaluation signals, in operation S750 it is determined whether the standard deviations of the second respiration evaluation signals are less than or equal to a product generated by multiplying the average of the standard deviations of the second respiration evaluation signals and ‘k.’ As a non-limiting example, a value that is in the predetermined range of the average may be 0.7, and ‘k’ may be used as a reference value for determining a sleep apnea section and may be 0.7. However, ‘k’ is not limited to 0.7 and may be any real number satisfying 0<k≦1.

If it is determined in operation S750 that the standard deviations of the second respiration evaluation signals are not less than or equal to a product of the average of the standard deviations of the second respiration evaluation signals and ‘k’, in S760 the second time intervals corresponding to the second evaluation signals are determined to be normal respiration estimation sections.

Alternatively, if it is determined in operation S750 that the standard deviations of the second respiration evaluation signals are less than or equal to a product of the average of the standard deviations of the second respiration evaluation signals and ‘k’, in S770 the second time intervals corresponding to the second evaluation signals are determined to be apnea estimation sections

In operation S780, a sleeping pattern is determined a number of times corresponding to the number of second evaluation signals.

Referring now to FIG. 7B, in operation S790, it is determined whether at least one of sleeping patterns for a plurality of second time intervals corresponding to the plurality of second evaluation signals includes an apnea estimation section. The number of apnea estimation sections is not limited.

If it is determined in operation S790 that at least one of the sleeping patterns includes an apnea estimation section, first time intervals corresponding to the first evaluation signals are determined to be sections in which sleep apnea occurs.

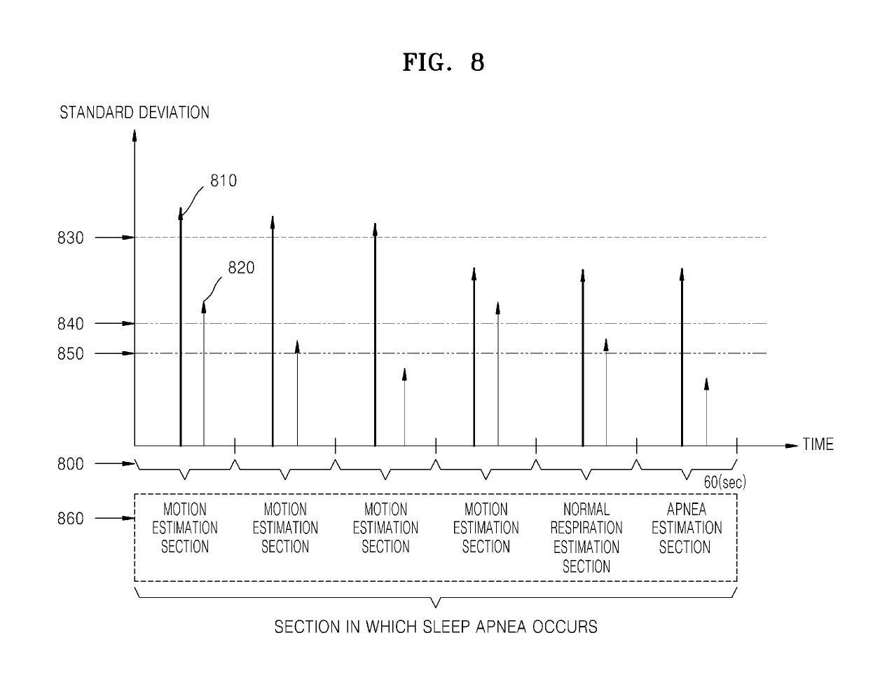

FIG. 8 is a diagram illustrating a section in which sleep apnea occurs, according to an exemplary embodiment.

In the example of FIG. 8, the X-axis represents time and the Y-axis represents standard deviations of second evaluation signals. The X-axis represents that a first time interval, e.g., one minute, may be divided by a second time interval 800, e.g., ten seconds. In this case, the first time interval may be divided into six sections 860, as illustrated in FIG. 8. The Y-axis represents a standard deviation 810 of second motion evaluation signals and a standard deviation 820 of second respiration evaluation signals in each of the six sections 860.

Sleeping patterns in the six sections 860 corresponding to the second time interval may be determined according to the values of the Y-axis (e.g., standard deviation information).

An average standard deviation 840 of the second respiration evaluation signals may be calculated based on the standard deviations 820 of the second respiration evaluation signals. In this example, a product 850 of the average standard deviation 840 and k may be also calculated to determine an apnea estimation section. For example, k=0.7 or may be a real number satisfying 0<k<1. A specific value 830 may also be used to determine a motion estimation section based on the standard deviations 810 of the second motion evaluation signals.

The average standard deviation 840 of the standard deviations 820 of the second respiration evaluation signals, excluding a maximum value among the standard deviations 820 of the second respiration evaluation signals calculated for a plurality of second time intervals may be calculated and determined as a reference value for determining a sleeping pattern. This is to prevent an average of all standard deviations from being influenced by a standard deviation in a section in which motion occurs.

In this example, in the first to third sections 860 among the six sections 860 of FIG. 8, the standard deviations 810 of the second motion evaluation signals are equal to or greater than the specific value 830 and sleeping patterns thereof is determined to be motion estimation sections.

In the fourth section 860, the standard deviation 810 of the second motion evaluation signals is less than the specific value 830 but the standard deviation 820 of the second respiration evaluation signals is greater than the average standard deviation 840 of the second respiration evaluation signals. Thus, a sleeping pattern in the fourth section 860 is determined to be a motion estimation section.

In the fifth section 860, the standard deviation 810 of the second motion evaluation signals is less than the specific value 830, but the standard deviation 820 of the second respiration evaluation signal is greater than the product 850 of the average standard deviation 840 and k. Thus, a sleeping pattern in the fifth section 860 is determined to be a normal respiration estimation section.

In the sixth section 860, the standard deviation 810 of the second motion evaluation signals is less than the specific value 830, and the standard deviation 820 of the second respiration evaluation signal is less than the product 850 of the average standard deviation 840 and k. Thus, a sleeping pattern in the sixth section 860 is determined to be an apnea estimation section. Because one apnea estimation section is present during the first time interval, e.g., one minute, as illustrated in FIG. 8, the sleeping pattern for the first time interval is determined to be a section in which sleep apnea occurs.

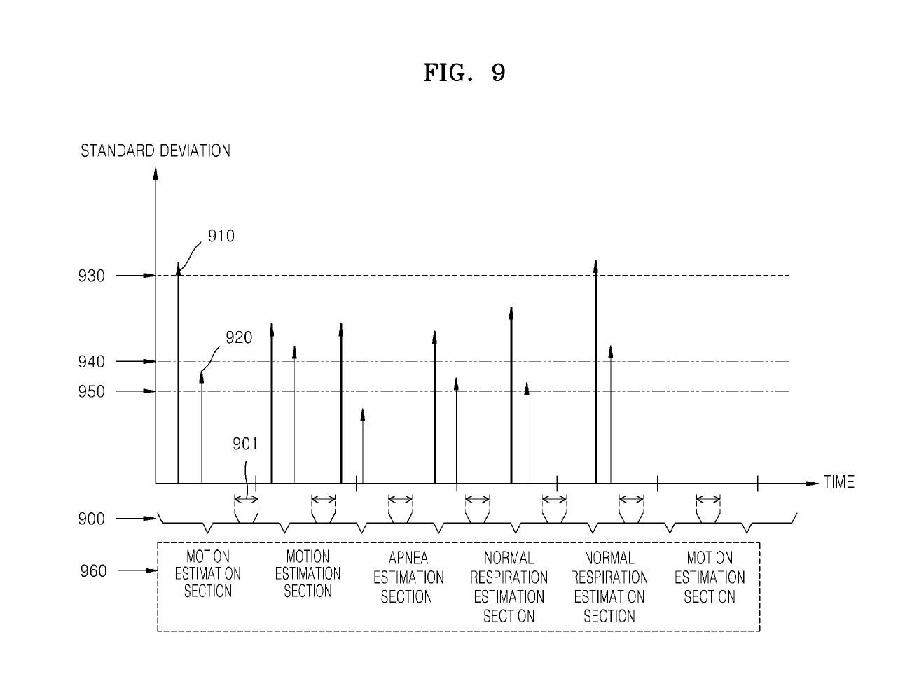

FIG. 9 is a diagram illustrating second evaluation signals that overlap with each other, according to an exemplary embodiment.

As illustrated in FIG. 9, a plurality of sections each having a second time interval 900 may overlap with each other.

For example, when a first evaluation signal is divided into sections such as 03:09:58 to 03:10:32, and 03:10:28 to 03:11:02, and the like, overlapping portions 901 between the sections is four seconds. The number of second evaluation signals generated from the first evaluation signal varies according to the overlapping portions 901. Thus, the number of sections generated for a first time interval may vary according to the number of the second evaluation signals.

FIG. 10 is a diagram illustrating a method of determining that apnea lasts, according to an exemplary embodiment.

As illustrated in FIG. 10, for example, when a minimum interval 1100 between second evaluation signals determined to be apnea estimation sections 1080 and 1090 is less than a second time interval, e.g., ten seconds, it may be determined that apnea lasts for the minimum interval 1100.

As described above, in a method of estimating sleep apnea, according to the one or more of the above exemplary embodiments, a bio-signal may be measured from a user in an unrestrictive manner during a sleep period without directly attaching sensors to the body of the user, thereby enabling it to determine whether sleep apnea occurs in a more convenient environment.

Also, sleep apnea may be estimated based on standard deviation information corresponding to the size of the bio-signal. Accordingly, sleep apnea estimation may be performed in a simple manner and at low costs.

A device for determining a sleeping pattern, according to an exemplary embodiment may include a processor, a memory that stores and runs program data, a permanent storage such as a disc drive, a user interface device, such as a touch panel, a key, a button, and the like.

Methods that can be embodied as a software module or an algorithm may be stored as computer readable codes or program commands, which may be executed by the processor, in a computer readable storage medium. Examples of the computer readable storage medium include magnetic storage media (e.g., ROM, RAM, floppy disks, hard disks, etc.) and optical recording media (e.g., CD-ROMs, or DVDs). The computer readable storage medium can be distributed among computer systems that are interconnected through a network to store and execute storage medium. The computer readable codes or program commands stored in the computer readable medium may be read by a computer and executed by the processor.

All documents including cited published literatures, patent applications, patents, etc. may be individually and definitely or in its entirety incorporated into the invention.

To help understand the present description, reference numerals are used in exemplary embodiments shown in the drawings and specific terms are used to explain the exemplary embodiments. However, the present description is not limited by the specific terms and may include various elements that are obvious to those of ordinary skill in the art.

The elements of the present description may be embodied as functional blocks and various processes. The functional blocks may be embodied as various types of hardware and/or software structures which execute specific functions. For example, the present description may employ direct circuit structures such as memories, processing, logics, or look-up tables which control one or more microprocessors or execute various functions through other controlling devices. The elements can be programmed as software or executed as software elements. Similarly, the elements may be written in a programming or scripting language such as C, C++, JavaScript, an assembler, or the like, including various types of algorithms which are realized through a combination of data structures, processes, routines, or other programming structures. Functional aspects can be embodied as an algorithm executed by one or more processors. The aspects may also employ related arts to set up an electronic environment, process signals, and/or process data. The terms such as “mechanism,” “element,” “means,” and “structure” may be widely used and are not limited to mechanical and physical structures. The terms may be understood as including a series of routines of software in leakage with processors.

Specific executions described herein are only examples and are not intended to limit the scope of the invention. For clarity, electronic structures, control systems, software, and other functional aspects of the control systems may be omitted. Also, a connection between elements via lines or connection members illustrated in the drawings exemplarily represent functional connections and/or physical or circuit connections and are thus replaceable or may be represented as additional various types of functional connections, physical connections, or circuit connections in a real device. Also, unless an element is particularly mentioned using terms such as “necessarily”, “importantly”, etc., the element may not be dispensable elements for applying the invention.

The term “the” and similar demonstrative terms used in the specification and in the claims should be understood as covering both singular and plural forms. If the term “range” is used, the range may be regarded as including any individual values belonging to the “range” unless mentioned otherwise. Unless an order of performing operations of a method is particularly described, the operations may be performed in an appropriate order. Thus, the embodiments are not limited by the order in which the operations are described. All examples or exemplary terms (e.g., “etc.”) used herein are simply selected to describe the exemplary embodiments. The scope of is not limited by the examples or the exemplary terms when they are not defined in the claims.

While one or more embodiments have been described with reference to the figures, it will be understood by those of ordinary skill in the art that various changes in form and details may be made therein without departing from the spirit and scope thereof as defined by the following claims.

What is claimed is:

1. A method of determining a sleeping pattern, the method comprising:
measuring a first bio-signal from a user in an unrestrictive manner;
extracting a second bio-signal including a motion signal and a respiration signal, based on the first bio-signal;
determining an apnea measurement reference signal including a respiration measurement signal and a motion measurement signal, based on the second bio-signal;
generating a first evaluation signal based on the apnea measurement reference signal and a first time interval, and generating a plurality of second evaluation signals by dividing the first evaluation signal by a second time interval, that is smaller than the first time interval;
calculating standard deviation information based on the second evaluation signals; and
determining a sleeping pattern for the second time interval corresponding to the second evaluation signals, based on the standard deviation information, the sleeping pattern comprising at least one of a motion estimation section, an apnea estimation section, and a normal respiration estimation section.

2. The method of claim 1, wherein the determining the apnea measurement reference signal comprises:
filtering the second bio-signal using a low-pass filter; and
determining the respiration measurement signal by analyzing principle components of the filtered signal, and determining the motion measurement signal by analyzing principle components of the second bio-signal.

3. The method of claim 1, wherein the generating the second evaluation signals comprises generating a plurality of second motion evaluation signals by dividing a first motion evaluation signal included in the first evaluation signal by the second time interval, and generating a plurality of second respiration evaluation signals by dividing a first respiration evaluation signal included in the first evaluation signal by the second time interval.

4. The method of claim 3, wherein the calculating the standard deviation information comprises:
calculating standard deviations of the plurality of second motion evaluation signals and the plurality of second respiration evaluation signals, respectively; and
calculating an average of the standard deviations of the plurality of second respiration evaluation signals, except for a maximum standard deviation of the plurality of second respiration evaluation signals.

5. The method of claim 4, wherein the determining the sleeping pattern for the second time interval corresponding to the second evaluation signals comprises:
determining the second time interval corresponding to the second evaluation signals to be the motion estimation section when the standard deviations of the second motion evaluation signals are equal to or greater than a specific value or when the standard deviations of the second respiration evaluation signals are equal to or greater than the average of the standard deviations of the second respiration evaluation signals;
determining the second time interval corresponding to the second evaluation signals to be the apnea estimation section when the second time interval is not the motion estimation section and when the standard deviations of the second respiration evaluation signals are less than or equal to a product of the average of the standard deviations of the second respiration evaluation signals and ‘k’, where k is a real number satisfying 0<k<1; and
determining the second time interval corresponding to the second evaluation signals to be the normal respiration estimation section when the second interval time is neither the motion estimation section nor the apnea estimation section.

6. The method of claim 1, further comprising determining the first time interval corresponding to the first evaluation signal to be a section in which sleep apnea occurs in response to at least one o second time interval of the first evaluation signal being determined to be the apnea estimation section.

7. The method of claim 1, wherein, when a minimum interval between the second evaluation signals determined to be the apnea estimation sections is less than the second time interval, the method further comprises determining that apnea lasts for the minimum interval.

8. The method of claim 1, wherein the generating of the plurality of second evaluation signals for the first evaluation signal comprises generating the plurality of second evaluation signals to overlap with each other.

9. The method of claim 1, wherein the first bio-signal is measured based on a polyvinylidene difluoride (PVDF)-based sensor that comprises piezoelectric characteristics.

10. The method of claim 1, wherein the standard deviation information comprises standard deviations of the second motion evaluation signals and an average standard deviation of second respiration evaluation signals included in the second evaluation signals of the first evaluation signal.

11. The method of claim 1, wherein the apnea reference signal is divided by the first interval to generate a plurality of first evaluation signals, and each of the divided first evaluations signals is divided by the second interval to generate a plurality of second evaluation signals for each first evaluation signal.

12. A device for determining a sleeping pattern, the device comprising:
a signal measuring sensor configured to measure a first bio-signal from a user in an unrestrictive manner;
a bio-signal extractor configured to extract a second bio-signal including a motion signal and a respiration signal, based on the first bio-signal;
a reference signal determiner configured to determine an apnea measurement reference signal including a respiration measurement signal and a motion measurement signal, based on the second bio-signal;
an evaluation signal generator configured to generate a first evaluation signal based on the apnea measurement reference and a first time interval, and generate a plurality of second evaluation signals by dividing the first evaluation signal by a second time interval, that is smaller than the first time interval;
a standard deviation information calculator configured to calculate standard deviation information based on the second evaluation signals; and
a sleeping pattern determiner configured to determine a sleeping pattern for the second time interval corresponding to the second evaluation signals, based on the standard deviation information, the sleeping pattern comprising at least one of a motion estimation section, an apnea estimation section, and a normal respiration estimation section.

13. The device of claim 12, wherein the reference signal determiner comprises:
a low-pass filter configured to filter the second bio-signal; and
a principle component analyzer configured to determine the respiration measurement signal by analyzing principle components of the filtered signal, and determine the motion measurement signal by analyzing principle components of the second bio-signal.

14. The device of claim 12, wherein the evaluation signal generator comprises:
a first time divider configured to generate a first motion evaluation signal included in the first evaluation signal by dividing the motion measurement signal by a first time interval, and generate a first respiration evaluation signal included in the first evaluation signal by dividing the respiration measurement signal by the first time interval; and
a second time divider configured to generate a plurality of second motion evaluation signals by dividing the first motion evaluation signal by a second time interval, and generate a plurality of second respiration evaluation signals by dividing the first respiration evaluation signal by the second time interval.

15. The device of claim 12, wherein the standard deviation information calculator is configured to calculate standard deviations of the plurality of second motion evaluation signals and the plurality of second respiration evaluation signals, respectively, and calculate an average of the standard deviations of the plurality of second respiration evaluation signals, except for a maximum standard deviation of the plurality of second respiration evaluation signals.

16. The device of claim 15, wherein the sleeping pattern determiner is configured to:
determine the second time interval corresponding to the second evaluation signals to be the motion estimation section when the standard deviations of the second motion evaluation signals are equal to or greater than a specific value or when the standard deviations of the second respiration evaluation signals are equal to or greater than the average of the standard deviations of the second respiration evaluation signals;
determine the sleeping pattern for the second time interval corresponding to the second evaluation signals to be the apnea estimation section when the sleeping pattern is not the motion estimation section and when the standard deviations of the second respiration evaluation signals are less than or equal to a product of the average of the standard deviations of the second respiration evaluation signals and ‘k’, where k is a real number satisfying 0<k<1; and
determine the sleeping pattern for the second time interval corresponding to the second evaluation signals to be the normal respiration estimation section when the sleeping pattern is neither the motion estimation section nor the apnea estimation section.

17. The device of claim 12, further comprising an apnea generation determiner configured to determine the first time interval corresponding to the first evaluation signal to be a section in which apnea occurs when at least one apnea estimation section is present in a second time interval of the first evaluation signal.

18. The device of claim 12, wherein, when a minimum interval between apnea estimation sections determined for the first time interval is equal to the second time interval, the sleeping pattern determiner determines the minimum interval to be the apnea estimation section.

19. The device of claim 12, wherein the evaluation signal generator is configured to generate the plurality of second evaluation signals to overlap with each other.

20. The device of claim 12, wherein the signal measuring sensor comprises a polyvinylidene difluoride (PVDF)-based sensor that comprises piezoelectric characteristics.

21. The device of claim 12, wherein the standard deviation information comprises standard deviations of the second motion evaluation signals and an average standard deviation of second respiration evaluation signals included in the second evaluation signals of the first evaluation signal.

22. A non-transitory computer-readable medium having recorded thereon a computer program that is executable by a computer to perform the method of claim 12.

23. An apparatus for determining sleep apnea occurring in a user, the apparatus comprising:
a signal measuring sensor configured to detect bio-signals from a user while the user sleeps, and comprising a polyvinylidene difluoride (PVDF)-based sensor including piezoelectric characteristics; and
a controller configured to determine sleep apnea occurring in the user based on the bio-signals detected by the signal measuring sensor.

24. The apparatus of claim 23, wherein the signal measuring sensor is configured to be installed in a space between a back of the user and a bed mattress.

25. The apparatus of claim 23, wherein the signal measuring sensor comprises a thickness of 0.1 mm or less.
